SEQUENCE LISTING (26 sequences)

SEQ ID NO: 1 - DNA, 1437 bp, synthetic construct
  source 1..1437: mol_type = other DNA
atgcccagtctcgacttcacctccaccatggcaggagacctctacagccctccgtcccccctgggggactccctgctggacagtcctctgtgtggagagctgatggaggaccttccagacatctcccagtcaatgggatttgttttccccgaataccagagcaacggttcagggtcagagagttctacagcgctggacaccttgactccggcctccagtccatcgtcggccgtgtgtggagctgcaccagaacctgaagaaggtctcaacctggagtgtcgtgtttgttcagacaaggcctcaggcttccactatggagtgcatgcatgtgaaggctgcaagggtttcttcaggaggaccatcaggctgaagctgaagtacgacaagtgtgacctcaagtgcaagatccaaaagaaaaaccgcaacaagtgccagtactgccgattccacaagtgcctgtctgtgggcatgtcccacaacgccattcggtttggtcggatgccacaggcggagaagctgaagctcaaggcagaaagcagaatggtggaaaaagacgtggagagccccctgctggccgaccacaaggttctggtcaggcagatccacgaagcctacatgaagaacttcaacatgaacaaggccaaagctcggctcatcctcacgggaaagaccagtaaaccgcctttcatcatccatgacatggagacgttccagctggcggagaagacgttagcggtccatatggtaaacggtgagcccccagatgctgagagcgctcctcggtgtggggatgtgttcgcaggtgtggtttgcggggagctggagcagagggaggccgaagcccggctcttccactgctgccagagcacttcagtggaaactgtgacagagctgacagagttcgctaaagcagtgccaggttttcaggatctggatctgaatgatcaggtgactttattaaagtatggcgttcatgaagccatcttcaccctgctggcttcatgcatgaacaaagatggcctcctggtggcccggggaggaggcttcatcacacgtgaattcctcaaaagcctccgtcgtccattaagcgacatgatggagccaaagtttcagtttgccactcgattcaactccctggagctggacgacagtgacctggccctgtttgtggctgccatcatctgctgtggagaccgtcccggactggtggacgttcctctggtggagcggctgcaagagagcattgtccaagcactacagctccacctgctggccaatcatcccgacaacaccttcctcttcccccggcttcttcagaaactggctgacctgcgggcactggtcactgagcatgctcagctcgtgcaggacatcaaaacaacggaggacacgtcactgcaccctctgctgcaggagatctacagagacatgtactga

SEQ ID NO: 2 - protein, 478 aa, synthetic construct
MPSLDFTSTMAGDLYSPPSPLGDSLLDSPLCGELMEDLPDISQSMGFVFPEYQSNGSGSESSTALDTLTPASSPSSAVCGAAPEPEEGLNLECRVCSDKASGFHYGVHACEGCKGFFRRTIRLKLKYDKCDLKCKIQKKNRNKCQYCRFHKCLSVGMSHNAIRFGRMPQAEKLKLKAESRMVEKDVESPLLADHKVLVRQIHEAYMKNFNMNKAKARLILTGKTSKPPFIIHDMETFQLAEKTLAVHMVNGEPPDAESAPRCGDVFAGVVCGELEQREAEARLFHCCQSTSVETVTELTEFAKAVPGFQDLDLNDQVTLLKYGVHEAIFTLLASCMNKDGLLVARGGGFITREFLKSLRRPLSDMMEPKFQFATRFNSLELDDSDLALFVAAIICCGDRPGLVDVPLVERLQESIVQALQLHLLANHPDNTFLFPRLLQKLADLRALVTEHAQLVQDIKTTEDTSLHPLLQEIYRDMY

SEQ ID NO: 3 - DNA, 21 bp, synthetic construct
  source 1..21: mol_type = other DNA
cgaagcttatgcccagtctcg

SEQ ID NO: 4 - DNA, 23 bp, synthetic construct
  source 1..23: mol_type = other DNA
cgggatccgtacatgtctctgta

SEQ ID NO: 5 - DNA, 22 bp, synthetic construct
  source 1..22: mol_type = other DNA
tcgctagttggcatcgtttatg

SEQ ID NO: 6 - DNA, 20 bp, synthetic construct
  source 1..20: mol_type = other DNA
cggaggttcgaagacgatca

SEQ ID NO: 7 - DNA, 24 bp, synthetic construct
  source 1..24: mol_type = other DNA
accagcggaactgtttattttgag

SEQ ID NO: 8 - DNA, 22 bp, synthetic construct
  source 1..22: mol_type = other DNA
gttgaagggattgaagtgaggc

SEQ ID NO: 9 - DNA, 22 bp, synthetic construct
  source 1..22: mol_type = other DNA
agataaagccaccgaccaaatg

SEQ ID NO: 10 - DNA, 23 bp, synthetic construct
  source 1..23: mol_type = other DNA
gcagattcaacttgtctccgatg

SEQ ID NO: 11 - DNA, 17 bp, synthetic construct
  source 1..17: mol_type = other DNA
cgctgcgtcgtttgtgc

SEQ ID NO: 12 - DNA, 18 bp, synthetic construct
  source 1..18: mol_type = other DNA
ctgccgtgaagaccctcc

SEQ ID NO: 13 - DNA, 20 bp, synthetic construct
  source 1..20: mol_type = other DNA
cgctgaagaacagggcaaga

SEQ ID NO: 14 - DNA, 19 bp, synthetic construct
  source 1..19: mol_type = other DNA
gccaccgctggataaacaa

SEQ ID NO: 15 - DNA, 19 bp, synthetic construct
  source 1..19: mol_type = other DNA
tcatagcgacccctcctgg

SEQ ID NO: 16 - DNA, 21 bp, synthetic construct
  source 1..21: mol_type = other DNA
gctgccacagtagcctttctt

SEQ ID NO: 17 - DNA, 20 bp, synthetic construct
  source 1..20: mol_type = other DNA
ggggctttcgtttccgttat

SEQ ID NO: 18 - DNA, 20 bp, synthetic construct
  source 1..20: mol_type = other DNA
gaggccgtgaggtcattagg

SEQ ID NO: 19 - DNA, 20 bp, synthetic construct
  source 1..20: mol_type = other DNA
tgtcaaagccatttccaccg

SEQ ID NO: 20 - DNA, 22 bp, synthetic construct
  source 1..22: mol_type = other DNA
cacaggaggagaaggaggattt

SEQ ID NO: 21 - DNA, 20 bp, synthetic construct
  source 1..20: mol_type = other DNA
tcgttggtggaaactggaag

SEQ ID NO: 22 - DNA, 19 bp, synthetic construct
  source 1..19: mol_type = other DNA
cccacagcaggctcaggat

SEQ ID NO: 23 - DNA, 19 bp, synthetic construct
  source 1..19: mol_type = other DNA
tgagtggcaccgtgtcgta

SEQ ID NO: 24 - DNA, 19 bp, synthetic construct
  source 1..19: mol_type = other DNA
ctcatggctcggagcttgt

SEQ ID NO: 25 - DNA, 20 bp, synthetic construct
  source 1..20: mol_type = other DNA
aggtgatgaaagccctgtgg

SEQ ID NO: 26 - DNA, 20 bp, synthetic construct
  source 1..20: mol_type = other DNA
cgtggacgaagcagttgatg

(19) United States
(12) Patent Application Publication
(10) Pub. No.: US 2024/0415926 A1
(43) Pub. Date: Dec. 19, 2024

(54) GENE PPARalpha FOR IMPROVING DISEASE RESISTANCE OF HALF-SMOOTH TONGUE SOLE CYNOGLOSSUS SEMILAEVIS AND USE THEREOF

(71) Applicant: Qingdao University, Qingdao (CN)

(72) Inventors: Zhenxia Sha, Qingdao (CN); Wenwen Wang, Qingdao (CN); Zhujun Li, Qingdao (CN); Xinbao Liu, Qingdao (CN); Hongning Liu, Qingdao (CN); Sen Han, Qingdao (CN); Zhendong Wu, Qingdao (CN); Jie Ma, Qingdao (CN); Suxu Tan, Qingdao (CN)

(21) Appl. No.: 18/611,711

(22) Filed: Mar. 21, 2024

(30) Foreign Application Priority Data
Jun. 19, 2023 (CN) 202310720402.8

(51) Int. Cl. A61K 38/17; A61P 31/04; C12N 15/63

(52) U.S. Cl. CPC A61K 38/1709; A61P 31/04; C12N 15/63

(57) ABSTRACT

The present invention belongs to the field of molecular biology technology, and specifically relates to the use thereof of the disease resistance gene PPARalpha and its encoded protein in half-smooth tongue sole (Cynoglossus semilaevis). The present invention provides the application of PPARalpha gene and protein in making products for preventing and treating fish bacterial diseases. The overexpression of PPARalpha gene significantly improves the anti-infection ability of half-smooth tongue sole, reduces the mortality after bacterial infection. In addition, it improves the oxidative stress level of fish and the ability to metabolize toxic substances, regulates apoptosis and energy metabolism pathways in the immune process. Therefore, the products containing PPARalpha gene can be used to prevent and treat bacterial diseases in fish and reduce economic losses caused by pathogenic bacterial infections in aquaculture.

### CROSS-REFERENCE TO RELATED APPLICATIONS

This application claims priority of Chinese Patent Application No. 202310720402.8, filed on Jun. 19, 2023, the entire contents of which are incorporated herein by reference.

### INCORPORATES BY REFERENCE OF SEQUENCE LISTING XML

A XML file is incorporates by reference. The file name is BJBYLJ-US-1-13.xml; the creation date is Mar. 20, 2024, and the size of the file is 25644 bytes.

### FIELD OF THE INVENTION

The present invention belongs to the field of molecular biology technology, and specifically relates to the use thereof of the disease resistance gene PPARalpha and its encoded protein in half-smooth tongue sole (Cynoglossus semilaevis).

### BACKGROUND

Half-smooth tongues sole (Cynoglossus semilaevis) is an important marine economically cultured fish along the northern and southeastern coasts of China. Vibrio disease is a serious bacterial disease in marine fish, and Vibrio anguillarum is one of the main pathogenic bacteria. As an opportunistic pathogen, V. anguillarum can infect fish, bivalves, crustaceans and other marine organisms, causing hemorrhagic septicemia and causing huge economic losses to the aquaculture industry, including half-smooth tongue sole aquaculture industry. Therefore, using molecular biology methods to study the function of disease resistance genes and their regulatory mechanisms in the interaction between fish and pathogenic bacteria is crucial to the cultivation of new disease-resistant fish varieties and the development of environmentally friendly and efficient disease-resistant fishery drugs.

Peroxisome proliferators-activated receptor (PPAR) belongs to the nuclear receptor C1 family in the steroid receptor superfamily. It is a ligand-activated receptor in the nuclear hormone receptor family and a ligand-dependent transcription factor. After activation, PPARalpha regulates the expression of a variety of nuclear target genes, and plays important roles in a series of life processes, such as sugar and lipid metabolism, insulin secretion and signal transduction, oxidative stress, cell growth and differentiation. PPARalpha is mainly expressed in the liver, heart, skeletal muscle, kidney and brain.

In mammals, after PPARalpha is activated, genes related to fatty acid transport, such as fatty acid transferase (FAT), acyl-CoA thioesterase 1, carnitine palmitoyltransferas 1a (CPT1a), and CPT1b, were significantly upregulated. In teleost, lipid metabolism processes are also regulated by PPARalpha. After PPARalpha activation, CPT1a, CPT1b, Acyl-CoA oxidase and Acyl-CoA carboxylase B oxidation were upregulated, thereby reducing the triglyceride content in the liver of bony fish.

So far, there are few studies on PPARalpha in the immune regulation of teleost.

### SUMMARY OF THE INVENTION

The technical problem to be solved by the present invention is to provide the application of the disease-resistant gene PPARalpha and its encoded protein in half-smooth tongue sole. The expression of the gene PPARalpha can prevent and treat fish bacterial diseases, improve the oxidative stress level of fish and the metabolic ability of toxic substances, regulate the expression of immunity-and metabolism-related genes and enhance fish immunity.

The present invention is achieved through the following technical solutions:

The present invention provides the application of the disease-resistant gene PPARalpha of half-smooth tongue sole in the preparation of products for preventing and treating fish bacterial diseases. The nucleotide sequence of the gene PPARalpha is shown in SEQ ID NO: 1.

The nucleotide sequence of the gene PPARalpha:

ATGCCCAGTCTCGACTTCACCTCCACCATGGCAGGAGACCTCTACAGCCC
TCCGTCCCCCCTGGGGGACTCCCTGCTGGACAGTCCTCTGTGTGGAGAGC
TGATGGAGGACCTTCCAGACATCTCCCAGTCAATGGGATTTGTTTTCCCC
GAATACCAGAGCAACGGTTCAGGGTCAGAGAGTTCTACAGCGCTGGACAC
CTTGACTCCGGCCTCCAGTCCATCGTCGGCCGTGTGTGGAGCTGCACCAG
AACCTGAAGAAGGTCTCAACCTGGAGTGTCGTGTTTGTTCAGACAAGGCC
TCAGGCTTCCACTATGGAGTGCATGCATGTGAAGGCTGCAAGGGTTTCTT
CAGGAGGACCATCAGGCTGAAGCTGAAGTACGACAAGTGTGACCTCAAGT
GCAAGATCCAAAAGAAAAACCGCAACAAGTGCCAGTACTGCCGATTCCAC
AAGTGCCTGTCTGTGGGCATGTCCCACAACGCCATTCGGTTTGGTCGGAT
GCCACAGGCGGAGAAGCTGAAGCTCAAGGCAGAAAGCAGAATGGTGGAAA
AAGACGTGGAGAGCCCCCTGCTGGCCGACCACAAGGTTCTGGTCAGGCAG
ATCCACGAAGCCTACATGAAGAACTTCAACATGAACAAGGCCAAAGCTCG
GCTCATCCTCACGGGAAAGACCAGTAAACCGCCTTTCATCATCCATGACA
TGGAGACGTTCCAGCTGGCGGAGAAGACGTTAGCGGTCCATATGGTAAAC
GGTGAGCCCCCAGATGCTGAGAGCGCTCCTCGGTGTGGGGATGTGTTCGC
AGGTGTGGTTTGCGGGGAGCTGGAGCAGAGGGAGGCCGAAGCCCGGCTCT
TCCACTGCTGCCAGAGCACTTCAGTGGAAACTGTGACAGAGCTGACAGAG
TTCGCTAAAGCAGTGCCAGGTTTTCAGGATCTGGATCTGAATGATCAGGT
GACTTTATTAAAGTATGGCGTTCATGAAGCCATCTTCACCCTGCTGGCTT
CATGCATGAACAAAGATGGCCTCCTGGTGGCCCGGGGAGGAGGCTTCATC
ACACGTGAATTCCTCAAAAGCCTCCGTCGTCCATTAAGCGACATGATGGA
GCCAAAGTTTCAGTTTGCCACTCGATTCAACTCCCTGGAGCTGGACGACA
GTGACCTGGCCCTGTTTGTGGCTGCCATCATCTGCTGTGGAGACCGTCCC
GGACTGGTGGACGTTCCTCTGGTGGAGCGGCTGCAAGAGAGCATTGTCCA
AGCACTACAGCTCCACCTGCTGGCCAATCATCCCGACAACACCTTCCTCT
TCCCCCGGCTTCTTCAGAAACTGGCTGACCTGCGGGCACTGGTCACTGAG
CATGCTCAGCTCGTGCAGGACATCAAAACAACGGAGGACACGTCACTGCA
CCCTCTGCTGCAGGAGATCTACAGAGACATGTACTGA.

The present invention also provides the application of the disease-resistant protein PPARalpha of half-smooth tongue sole in the preparation of products for preventing and treating fish bacterial diseases. The amino acid sequence of the protein PPARalpha is shown in SEQ ID NO: 2.

The amino acid sequence of the protein PPARalpha:

MPSLDFTSTMAGDLYSPPSPLGDSLLDSPLCGELMEDLPDISQSMGFVFP
EYQSNGSGSESSTALDTLTPASSPSSAVCGAAPEPEEGLNLECRVCSDKA
SGFHYGVHACEGCKGFFRRTIRLKLKYDKCDLKCKIQKKNRNKCQYCRFH
KCLSVGMSHNAIRFGRMPQAEKLKLKAESRMVEKDVESPLLADHKVLVRQ
IHEAYMKNFNMNKAKARLILTGKTSKPPFIIHDMETFQLAEKTLAVHMVN
GEPPDAESAPRCGDVFAGVVCGELEQREAEARLFHCCQSTSVETVTELTE
FAKAVPGFQDLDLNDQVTLLKYGVHEAIFTLLASCMNKDGLLVARGGGFI
TREFLKSLRRPLSDMMEPKFQFATRFNSLELDDSDLALFVAAIICCGDRP
GLVDVPLVERLQESIVQALQLHLLANHPDNTFLFPRLLQKLADLRALVTE
HAQLVQDIKTTEDTSLHPLLQEIYRDMY.

The products have at least one of the following functions from (1) to (3):

Regulate the Toll-like receptor and JAK-STAT signaling pathways;

Improve the antioxidant capacity of fish and ability to metabolize toxic substances

Regulate the metabolic pathways of carbohydrates and lipids in fish.

The effective ingredient of the product is the disease-resistant protein PPARalpha of half-smooth tongue sole or the recombinant expression plasmid containing the gene PPARalpha. The products include feed, feed additives, pharmaceutical compositions

The method for preparing a recombinant expression plasmid containing the gene PPARalpha, including the following steps:

Extract total RNA from half-smooth tongue sole and reverse-transcribe to obtain total cDNA;

Design the forward primer and reverse primer of the gene PPARalpha, and obtain the amplification product using the total cDNA of half-smooth tongue sole as a template.

Molecularly clone the amplification product to obtain a recombinant expression plasmid;

The enzyme cleavage sites of molecular cloning are HindIII and BamHI;

The sequence of forward primer is CGAAGCTTATGCCCAGTCTCG, as shown in SEQ ID NO: 3;

The sequence of reverse primer is CGGGATCCGTACATGTCTCTGTA, as shown in SEQ ID NO: 4.

Compared with the existing technology, the present invention has the following beneficial effects:

The overexpression of PPARalpha gene significantly improves the anti-infection ability of half-smooth tongue sole, reduces the mortality after bacterial infection. In addition, it improves the oxidative stress level of fish and the ability to metabolize toxic substances, regulates apoptosis and energy metabolism pathways in the immune process. Therefore, the products containing PPARalpha gene can be used to prevent and treat bacterial diseases in fish, and reduce economic losses caused by pathogenic bacterial infections in aquaculture, which is crucial for the development of aquaculture industry.

### BRIEF DESCRIPTION OF THE DRAWINGS

FIG. 1. Schematic diagram of the structure of pEGFP-N1-CsPPARalpha recombinant overexpression plasmid.

FIG. 2. Amplification electropherogram of PPARalpha gene.

FIG. 3. Electropherogram of double restriction-enzyme digestion produces of PEGFP-N1-CsPPARalpha recombinant overexpression plasmid.

FIG. 4. Survival analysis curve of half-smooth tongue sole infected with Vibrio anguillarum.

FIG. 5. The expression profiles of differentially expressed genes related to Toll-like receptor and JAK-STAT3 signaling pathways after PPARalpha overexpression.

FIG. 6. Eenriched pathways of differentially expressed genes between control group (C) and PPARalpha overexpression group (P.

FIG. 7. Enriched pathways of differentially expressed genes between Vibrio anguillarum infection group (V) and the group of V. anguillarum infection after PPARalpha overexpression (PV).

FIG. 8. Enriched pathways of differential metabolites between control group (C) and PPARalpha overexpression group (P).

FIG. 9. Enriched pathways of differential metabolites between Vibrio anguillarum infection group (V) and the group of V. anguillarum infection after PPARalpha overexpression (PV).

FIG. 10. The qRT-PCR results of significantly differentially expressed genes after PPARalpha overexpression.

### DETAILED DESCRIPTION

The following examples further describe the present invention. It should be noted that all materials used in the examples are commercially available unless otherwise stated. The primers were synthesized by Beijing Tsingke Biotech Co., Ltd., CN; SYBR Green Mix was purchased from Nanjing vazyme Biotech Co., Ltd., CN; RNA-sequencing and metabolome detection were completed by Shanghai Bioprofile Technology Company, Ltd., CN.

### Example 1

Preparation of PPARalpha recombinant expression plasmid pEGFP-N1-CsPPARalpha, including the following steps:

Extract total RNA from half-smooth tongue sole and reverse-transcribe to obtain total cDNA;

(1) Design the forward primer and reverse primer of the gene PPARalpha, and obtain the amplification product using the total cDNA of half-smooth tongue sole as a template.

The sequence of forward primer is CGAAGCTTATGCCCAGTCTCG, as shown in SEQ ID NO: 3;

The sequence of reverse primer is CGGGATCCGTACATGTCTCTGTA, as shown in SEQ ID NO: 4.

PCR reaction system: 2×Taq enzyme mix 20 μL, forward primer 2 μL, reverse primer 2 μL, cDNA 2 μL, ddH₂O 14 μL; PCR amplification program: pre-denaturation at 95° C. for 5 minutes, 95° C. for 15 seconds, 60° C. for 30 seconds, 72° C. for 90 seconds, 35 cycles;

(1) Molecularly clone the amplification product to obtain a recombinant expression plasmid;

Using HindIII and BamHI as enzyme cleavage sites, the amplification product PPARalpha was inserted into the pEGFP-NI vector to construct the pEGFP-N1-CsPPARalpha recombinant expression plasmid (FIG. 1).

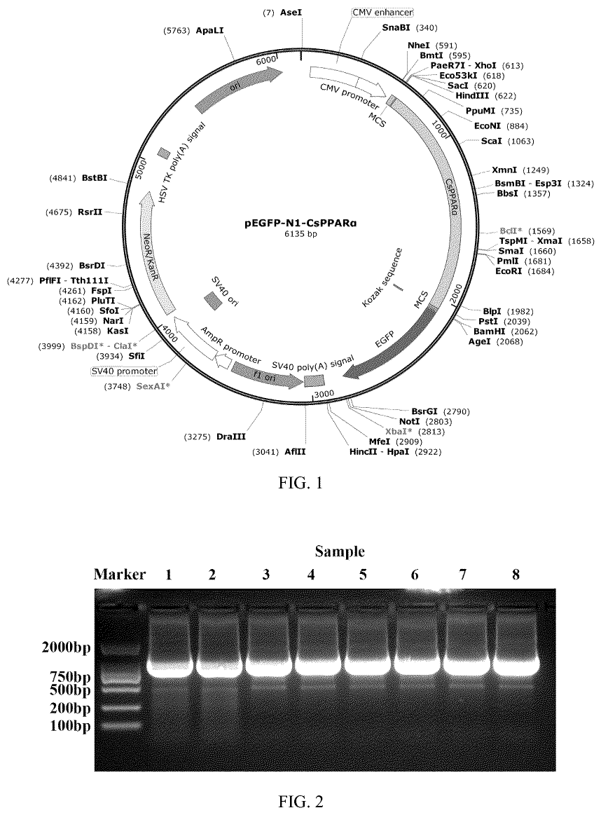

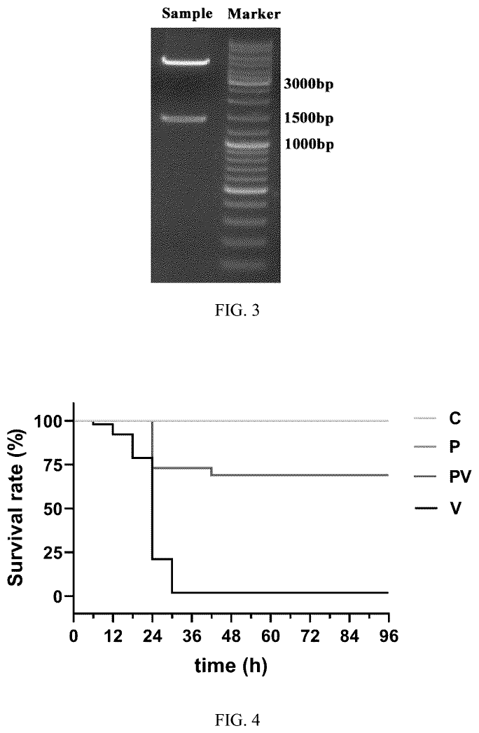

As shown in FIG. 2, 1-8 are the PCR amplification products of the gene PPARalpha. The bands are clear (1437 bp), indicating that the gene PPARalpha was successfully amplified. The recombinant expression plasmid was double restriction-enzyme digested, and then subjected to agarose gel electrophoresis. As shown in FIG. 3, two enzyme digestion products can be clearly seen in the left band, and the size of the small fragment of the enzyme digestion product is consistent with the size of the gene PPARalpha, indicating that PPARalpha was successfully inserted into the pEGFP-NI vector.

Example 2

The half-smooth tongue sole used in the experiment has a weight of 6.0+2.0 g and a body length of 9.0+1.5 cm. They were cultured in 56 cm×45 cm×32 cm tanks with 30 L of seawater. The water temperature was 24+1° C., the salinity was 30% 0, the dissolved oxygen content was 8.0=0.5 mg/L, and the pH was 8.0+0.2.

There were four groups, control group (C), PPARalpha overexpression group (P), V. anguillarum infection group (V) and V. anguillarum infection after PPARalpha overexpression (PV). Firstly, 1×PBS buffers (20 μL/each fish) were intravenously injected into the fish of C and V groups, and the PPARalpha overexpression plasmids (2 μg/mL, 20 μL/each fish) were injected into the fish of P and PV groups. Then, after 48 hours, V. anguillarum bacterial fluids (5×10⁵ CFU/mL, 50 μL/each fish) were intraperitoneally injected into the fish in V and PV groups, and 1×PBS buffers (50 μL/each fish) were intraperitoneally injected into the fish in C and P groups.

The numbers of deaths at 6, 12, 18, 24, 30, 36, 42, 48, 72, 84 and 96 h after bacterial infection were recorded, and the survival curve was plotted (FIG. 4). The mortalities in C and P groups were 0. In V and PV groups, the concentrated death time occurred at 12-24 hours after bacterial infection, and the mortality at 24 hours was 80% and 28%, respectively. Overexpression of PPARalpha could significantly improve the survival rate and the antibacterial infection ability of half-smooth tongue sole after V. anguillarum infection.

### Example 3

The fish liver of C, P, V, and PV groups after bacterial infection from the Example 2 were collected for further study.

1. Analyses of RNA-seq and non-targeted metabolome

(1) PPARalpha regulates the Toll-like receptor and JAK-STAT signaling pathways

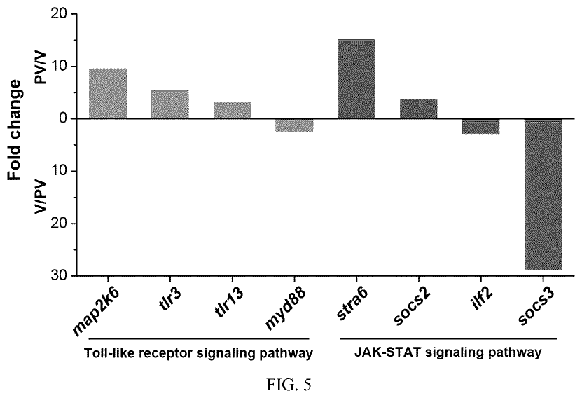

After PPARalpha was overexpression, genes of mitogen-activated protein kinase 6 (map2k6), toll-like receptor 3 (tlr3), toll-like receptor 13 (tlr13), MYD88 innate immune signal transduction adaptor (myd88), signaling receptor and transporter of retinol STRA6 (stra6), suppressor of cytokine signaling 2 (socs2), suppressor of cytokine signaling 3 (socs3), and interleukin enhancer-binding factor 2 (ilf2) were significantly differentially expressed, and enriched in Toll-like receptor and JAK-STAT signaling pathways (FIG. 5), which are important immunity and inflammation pathways. The results indicated that PPARalpha could regulate these pathways to improve the antibacterial infection ability of teleost.

(2) PPARalpha regulates abilities of antioxidant and detoxification

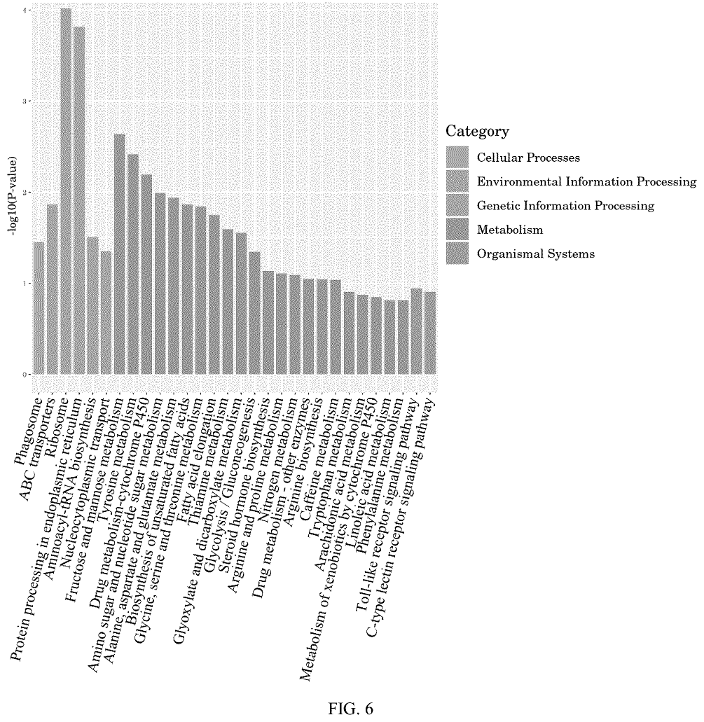

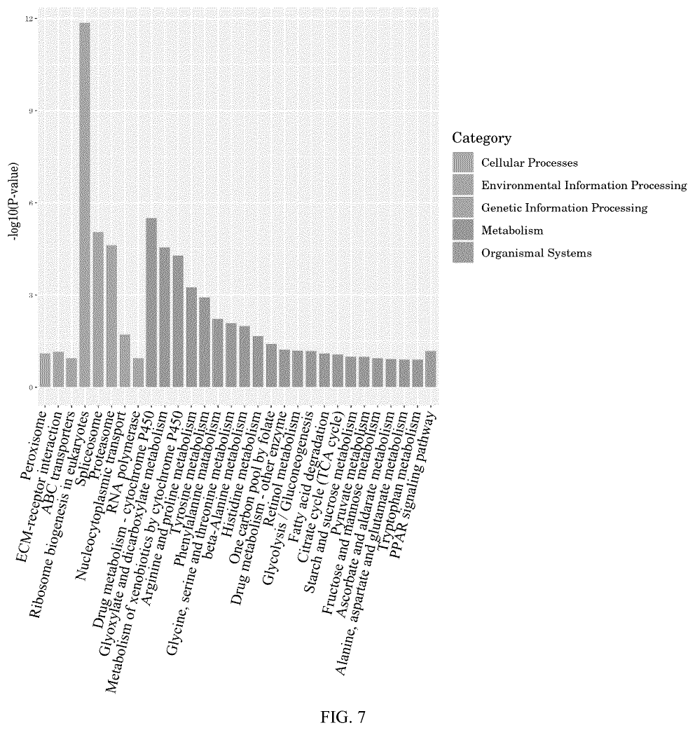

After PPARalpha overexpression, differentially expressed genes were enriched in the peroxisome pathway and drug metabolism cytochrome P450 pathway (FIG. 6 and FIG. 7). Among the differentially expressed genes enriched in the peroxisome pathway, most were upregulated, such as peroxiredoxin 2 (Prx2), superoxide dismutase 1 (SOD1). SOD and Prx are both important endogenous antioxidants that protect cells from oxidative stress damage. The differentially expressed genes enriched in the drug metabolism cytochrome P450 pathway were all upregulated, such as UDP-glucuronosyltransferase 2A1-like (UGT2A1-like), glutathione S-transferase rho (GST-R), and monoamine oxidase (MAO). UGT can catalyze the biotransformation of various substrates and participate in the most important second stage detoxification reactions in mammals and fish. GST is an enzyme that plays a crucial role in the detoxification process, catalyzing the reduction reaction of peroxides, promoting the thiol group of reduced glutathione (GSH) to bind with toxins, transforming hydrophobic toxic substances into hydrophilic substances, promoting their excretion in urine or bile, protecting the body from toxic damage, thereby improving the survival rate of organisms and resistance to stress factors. MAO is a mitochondrial marker, a key enzyme in the breakdown and metabolism of monoamine substances, and an enzyme that protects animals from oxidative stress, and is related to the immune response of vertebrates. The results indicated that overexpression of PPARalpha improved the antioxidant and detoxification abilities of half-smooth tongue sole.

(3) PPARalpha regulates carbohydrate and lipid metabolism pathways

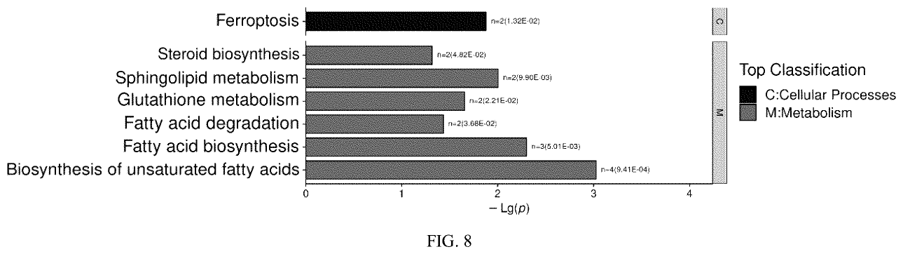

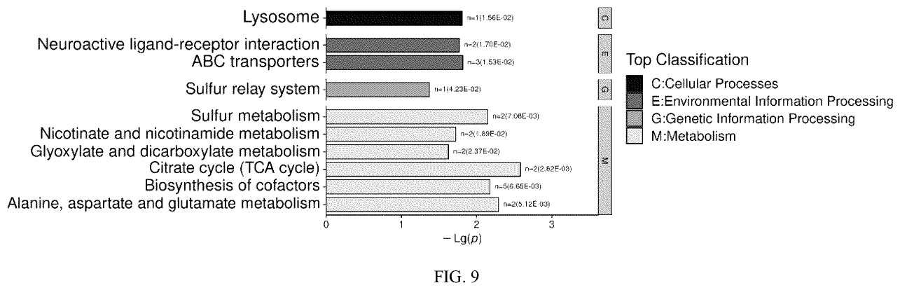

After PPAR overexpression, differentially expressed genes were also significantly enriched in glycolysis/gluconeogenesis signaling pathways and lipid metabolism pathways (FIG. 8 and FIG. 9). The differentially expressed genes enriched in the glycolysis/gluconeogenesis pathway included fructose-bisphosphate aldolase b (ALDOB), triosephosphate isomerase 1b (TIM1b), glyceraldehyde-3-phosphate dehydrogenase (GAPDH), dihydrolipoamide dehydrogenase (DLD), Phosphofructokinase-liver a (PFK-1a), fructose-1,6-bisphosphatase 1b (FBP1b), and pyruvate carboxylase b (PCb). In the pathways related to lipid metabolism, Elove5, Elov16, Elov17 involved in fatty acid synthesis and elongation, acyl-coAthioesterase 1 (ACOT1), and fatty acid synthase (FASN) were significantly down-regulated. Genes related to fatty acid degradation, such as carnitine palmitoyltransferas la, (CPT1b), Acyl-CoA oxidase (ACOX) were upregulated. In addition, overexpression of PPAR significantly increased the content of antibacterial fatty acids in liver tissue, such as oleic acid, stearic acid, and palmitic acid.

In summary, PPARalpha enhances the immunity and disease resistance by regulating the Toll-like receptor and JAK-STAT immune signaling pathways, regulating antioxidant and detoxification abilities, as well as regulating carbohydrate and lipid metabolism pathways.

2. The transcriptional expression of catalase (CAT), superoxide dismutase (SOD), glutathione s-transferase (GST-R), and nuclear factor-κB (NF-κB) were verified by qRT-PCR.

qPCR reaction system: 2×SYBR mix 10 μL, forward primer 0.4 μL, reverse primer 0.4 μL, cDNA 1 μL, ddH₂O 8.2 μL. The sequences of primers are shown in Table 1.

PCR amplification program: pre-denaturation at 95° C. for 30 seconds, 95°° C. for 10 seconds, 60° C. for 30 seconds, 40 cycles.

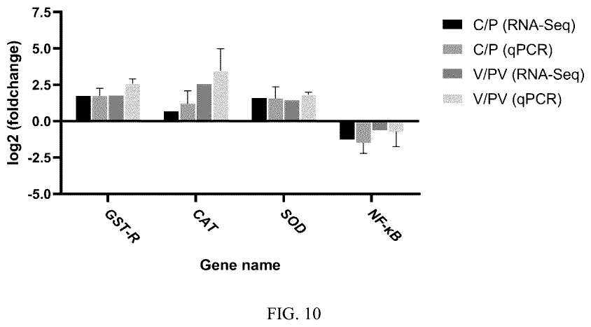

As shown in FIG. 10, the results of qRT PCR are consistent with the results of RNA-sequencing. CAT participates in clearing H₂O₂ from the body. SOD acts as a free radical scavenger, disproportionating superoxide anion radicals into H₂O₂ and O₂. CAT further decomposes cytotoxic H₂O₂ into H₂O and O₂, protecting cells from oxidative damage. GST is a key enzyme in the process of biological detoxification, promoting the thiol group of reduced glutathione (GSH) to bind with toxins, transforming hydrophobic toxic substances into hydrophilic substances, promoting their excretion in urine or bile, protecting the body from toxic damage, thereby improving the survival rate of organisms and their resistance to stress factors. NF-κB, as a mediator of pro-inflammation, plays a role in both innate and adaptive immunity. In this study, after PPARalpha overexpression, the expression levels of genes such as CAT, SOD, and GST-R were upregulated, and NF-κB was downregulated, confirming that PPARalpha plays an important role in improving the antioxidant and detoxification abilities in fish, as well as inhibiting inflammatory reactions.

TABLE 1
The sequence of primers used for qRT-PCR

| Primer | Sequence (5′-3′) |
|---|---|
| 18S-qF | TCGCTAGTTGGCATCGTTTATG |
| 18S-qR | CGGAGGTTCGAAGACGATCA |
| SOD-qF | ACCAGCGGAACTGTTTATTTTGAG |
| SOD-qR | GTTGAAGGGATTGAAGTGAGGC |
| CAT-qF | AGATAAAGCCACCGACCAAATG |
| CAT-qR | GCAGATTCAACTTGTCTCCGATG |
| UGT-qF | CGCTGCGTCGTTTGTGC |
| UGT-qR | CTGCCGTGAAGACCCTCC |
| GSTR-qF | CGCTGAAGAACAGGGCAAGA |
| GSTR-qR | GCCACCGCTGGATAAACAA |
| MAO-qF | TCATAGCGACCCCTCCTGG |
| MAO-qR | GCTGCCACAGTAGCCTTTCTT |
| NFκB-qF | GGGGCTTTCGTTTCCGTTAT |
| NFκB-qR | GAGGCCGTGAGGTCATTAGG |
| FBP-qF | TGTCAAAGCCATTTCCACCG |
| FBP-qR | CACAGGAGGAGAAGGAGGATTT |
| TIM-qF | TCGTTGGTGGAAACTGGAAG |
| TIM-qF | CCCACAGCAGGCTCAGGAT |
| ACOX1-qF | TGAGTGGCACCGTGTCGTA |
| ACOX1-qR | CTCATGGCTCGGAGCTTGT |
| Elov15-qF | AGGTGATGAAAGCCCTGTGG |
| Elov15-qR | CGTGGACGAAGCAGTTGATG |

We claim:

1. A use of the disease-resistant gene PPARalpha of half-smooth tongue sole in preparation of products for the prevention and treatment of fish bacterial diseases, wherein the fish is half-smooth tongue sole;
wherein the bacterial disease is Vibrio anguillarum infection;
wherein the nucleotide sequence of the gene PPARalpha is as SEQ ID NO.: 1 shown.

2. A use of the disease-resistant protein PPARalpha of half-smooth tongue sole in preparation of products for the prevention and treatment of fish bacterial diseases, wherein the fish is half-smooth tongue sole;
wherein the bacterial disease is Vibrio anguillarum infection;
wherein the amino acid sequence of the protein PPARalpha is shown in SEQ ID NO: 2.
